(19) United States
(12) Patent Application Publication
(10) Pub. No.: US 2023/0115554 A1
(43) Pub. Date: Apr. 13, 2023

(54) TECHNOLOGY ADAPTED FOR IMPROVED ASSESSMENT OF COGNITIVE FUNCTION IN A HUMAN SUBJECT, INCLUDING ASSESSMENT OF COGNITIVE FUNCTION AFFECTED BY BRAIN INJURIES SUSTAINED DURING SPORTING ACTIVITIES

(71) Applicant: HitlQ Limited, South Melbourne, VIC (AU)

(72) Inventors: Mike Vegar, Qucenscliff, NSW (AU); Ben Nizette, Quceenscliff, NSW (AU); Thomas Rau, Quceenscliff, NSW (AU); Andrew Gardner, Quceenscliff, NSW (AU)

(21) Appl. No.: 17/906,042

(22) PCT Filed: Mar. 10, 2021
(86) PCT No.: PCT/AU2021/050211

§ 371 Date: Sep. 9, 2022

(30) Foreign Application Priority Data
Mar. 10, 2020 (AU) 2020900734

(51) Int. Cl. A61B 5/00; A61B 5/16; G06F 3/01

(52) U.S. Cl. CPC A61B 5/4064; A61B 5/163; A61B 5/682; A61B 5/4023; A61B 5/6803; G06F 3/011; A61B 2503/10

(57) ABSTRACT

Assessment of cognitive function affected by brain injuries is achieved. This included, but is not limited to, assessment of cognitive function affected by brain injuries, for example injuries sustained during sporting activities. A virtual reality system is used to apply controlled cognitive loading to the subject, via a series of distinct test types which in combination apply an increasing cognitive load over time. Results are optionally assessed in conjunction with data from an instrumented mouthguard and/or Finite Element Analysis (FEA) model.

### FIELD OF THE INVENTION

The present invention relates, in various embodiments, to technology adapted for improved assessment of cognitive function in a human subject. This included, but is not limited to, assessment of cognitive function affected by brain injuries, for example injuries sustained during sporting activities. In some embodiments a virtual reality system is used to apply controlled cognitive loading to the subject, via a series of distinct test types which in combination apply an increasing cognitive load over time. Results are optionally assessed in conjunction with data from an instrumented mouthguard and/or Finite Element Analysis (FEA) model. While some embodiments will be described herein with particular reference to those applications, it will be appreciated that the invention is not limited to such a field of use, and is applicable in broader contexts.

### BACKGROUND

Any discussion of the background art throughout the specification should in no way be considered as an admission that such art is widely known or forms part of common general knowledge in the field.

Brain injuries, particularly those sustained during participation in contact sports, are becoming an increasingly important focus of attention. For example, head impacts sustained during sport can have serious effects of both short term and long-term participant welfare. For example, it is valuable to better understand the nature of a suspected brain injury in terms of: (i) whether a participant should be rested from participation; (ii) an extent to which the injury should prevent a return to activity; (iii) a degree of seriousness of an injury, for instance insofar as that might affect treatment and management; and (iv) better understanding cumulative effects of successive brain injuries for a given participant.

It is known to use virtual reality technology to assess brain injuries. These include systems which make use of a modified virtual reality system with eye movement tracking capabilities thereby to track eye movement in response to stimuli. This in a way provides a technology-based solution for mimicking known gaze-based assessments. An advantage of these systems is that testing is able to be performed at a wide range of locations (for example “sideline testing”, without a need for clinical experts with special skills in delivering testing. However, in focussing on attributes such as reaction time and gaze smoothness, there is a limited depth of information regarding a potential brain injury that is able to be gained.

### SUMMARY OF THE INVENTION

It is an object of the present invention to overcome or ameliorate at least one of the disadvantages of the prior art, or to provide a useful alternative.

Example embodiments are described below in the section entitled “claims”.

One embodiment provides a computer implemented method configured to enable assessment of a brain injury or other physiological condition, the method including:
maintaining access to computer executable code representative of a plurality of neurological tests that are renderable via hardware including a virtual reality system, wherein the plurality of neurological tests includes neurological tests belonging to a plurality of distinct test classes;
configuring the virtual reality system to deliver, to a subject, using the virtual reality system, a neurological assessment including a sequence constructed from the plurality of neurological tests, wherein the sequence of the neurological tests is defined thereby to sequentially provide tests belonging to different ones of the plurality of distinct test classes, thereby to deliver an increasing cognitive load;
obtaining subject performance data representative of performance of the subject in the neurological assessment; and
processing the subject performance data thereby to derive one or more measures representative of subject neurological conditions.

One embodiment provides a method wherein the processing the subject performance data includes identifying variations in performance attributable to increasing of cognitive loading.

One embodiment provides a method identifying variations in performance attributable to increasing of cognitive loading includes comparing subject performance with a plurality of tests belonging to a particular one of the distinct test classes which are delivered non-adjacently with respect to the sequence.

One embodiment provides a method wherein identifying variations in performance attributable to increasing of cognitive loading includes comparing subject performance with a first test belonging to a particular one of the distinct test classes with a second test belonging to the same particular one of the distinct test classes, wherein the second test is delivered subsequent to the first test non-adjacently with respect to the sequence.

One embodiment provides a method wherein the plurality of distinct test classes includes one or more a test classes defined by defined forms of memory test.

One embodiment provides a method wherein the defined forms of memory test includes: immediate memory, working memory, and delayed memory tests.

One embodiment provides a method wherein the plurality of distinct test classes include a class defined by a defined form of short-term memory test.

One embodiment provides a method wherein the defined form of short-term memory test includes a list item recollection exercise delivered via the virtual reality system.

One embodiment provides a method wherein the plurality of distinct test classes include a class defined by a defined form of long-term memory test.

One embodiment provides a method wherein the defined form of memory test includes a list item recollection exercise delivered via the virtual reality system, wherein the list is presented preceding one or more tests of other test classes, and recollection tested following the one or more tests of other test classes.

One embodiment provides a method wherein the plurality of distinct test classes include a class defined by a defined form of vestibular system test.

One embodiment provides a method wherein the defined form of vestibular system test includes a virtual reality game for which performance is related to gaze control and/or saccades, wherein the subject stands on a computerised balance board during the test.

One embodiment provides a method wherein the plurality of distinct test classes include a class defined by a defined form of reaction time test.

One embodiment provides a method wherein the defined form of reaction time test includes an auditory reaction time test.

One embodiment provides a method wherein the plurality of distinct test classes include a class defined by a defined form of ocular reaction time test.

One embodiment provides a method wherein the defined form of ocular reaction time test includes a test in which the VR system displays to the subject a moving object, and the subject is instructed to track that object with a stationary head, and provide a defined input upon the object performing a specified change in behaviour.

One embodiment provides a method wherein the plurality of distinct test classes include a class defined by a defined form of vestibular system test in which the subject is instructed to shift balance on an computerised balance board.

One embodiment provides a method wherein the plurality of distinct test classes include a class defined by a defined form of executive cognitive function test.

One embodiment provides a method wherein the sequence constructed from a subset of the plurality of neurological tests is a sequence which includes a sub-sequence including three or more of the following thereby to increase cognitive loading through the sub-sequence: a memory test; a vestibular system test; a reaction time test; and an executive cognitive function test.

One embodiment provides a method wherein the sequence is selected based on input data representative of an observed or suspected traumatic event involving the subject.

One embodiment provides a method wherein the input data representative of an observed or suspected traumatic event involving the subject includes input data based on measurements made by an instrumented mouthguard device.

One embodiment provides a method wherein the input data representative of an observed or suspected traumatic event involving the subject includes input data based on operation of a computerised brain model

One embodiment provides a method wherein the computerised brain model is a Finite Element Analysis (FEA) model.

One embodiment provides a method wherein the FEA model is configured to operate based on input device from an instrumented mouthguard device.

One embodiment provides a method wherein the one or more measures representative of subject neurological conditions are combined with data derived from instrumented observation of a traumatic event.

One embodiment provides a method wherein the instrumented observation of a traumatic event is observed via an instrumented mouthguard device.

One embodiment provides a method wherein the data derived from instrumented observation of a traumatic event includes output from a computerised brain model.

One embodiment provides a method wherein the computerised brain model is a Finite Element Analysis (FEA) model.

One embodiment provides a method wherein the FEA model is configured to operate based on input device from an instrumented mouthguard device.

One embodiment provides a method wherein the one or more measures representative of subject neurological conditions are compared with benchmarked measures representative of subject neurological conditions for the subject.

One embodiment provides a method for assessing a brain injury, the method including:
accessing a first data set representative of an observed traumatic event, wherein the first data set is generated in response to data derived from one or more subject-worn motion sensors;
accessing a second data set representative of neurological conditions following the observed traumatic event, wherein the second data set is generated in response to data derived from subject performance data in a neurological assessment delivered by a virtual reality system;
processing a combination of data from the first data set and the second data set thereby to define a third data set representative of an enhanced brain injury assessment.

One embodiment provides a method wherein the one or more subject-worn motion sensors are provided by an instrumented mouthguard device.

One embodiment provides a method wherein the first data set includes a metric derived from processing of data provided by the instrumented mouthguard device.

One embodiment provides a method wherein the first data set includes output from a brain model that is executed base on the data derived from one or more subject-worn motion sensors.

One embodiment provides a method wherein the brain model is a FEA model.

One embodiment provides a method wherein processing a combination of data from the first data set and the second data set includes identifying a correlation between an output of the FEA model and performance in the neurological assessment.

One embodiment provides a method wherein identifying a correlation between an output of the FEA model and performance in the neurological assessment includes benchmarking against prior results for different subjects.

One embodiment provides a method wherein identifying a correlation between an output of the FEA model and performance in the neurological assessment includes benchmarking against prior results for the same subject.

One embodiment provides a method wherein the third data set includes a metric representative of a deviation between: (i) expected performance in the neurological assessment based on the observed traumatic event; and (ii) actual performance in the neurological assessment based on the observed traumatic event.

One embodiment provides a method wherein the third data set is used to test and/or validate, via the second data set, a hypothesis as to the nature of a brain injury made based on the first data set.

One embodiment provides a method for assessing a brain injury, the method including:
accessing a first data set representative of an observed traumatic event, wherein the first data set is generated in response to data derived from one or more subject-worn motion sensors;
based on the first data set, configuring a virtual reality system to deliver a neurological assessment having defined parameters to the subject, and in response define a second data set representative of subject performance in the assessment; and
performing a brain injury assessment based on a combination of the first data set and the second data set.

One embodiment provides a method wherein the one or more subject-worn motion sensors are provided by an instrumented mouthguard device.

One embodiment provides a method wherein the first data set includes a metric derived from processing of data provided by the instrumented mouthguard device.

One embodiment provides a method wherein the first data set includes output from a brain model that is executed base on the data derived from one or more subject-worn motion sensors.

One embodiment provides a method wherein the brain model is a FEA model.

One embodiment provides a method wherein the neurological assessment has one or more parameters selected based on an output of the FEA model

One embodiment provides a method wherein the one or more parameters include a sequencing of sub-tests belonging to distinct classes.

One embodiment provides a method including identifying a correlation between an output of the FEA model and performance in the neurological assessment.

One embodiment provides a method including defining a measure representative of a deviation between: (i) expected performance in the neurological assessment based on the observed traumatic event; and (ii) actual performance in the neurological assessment based on the observed traumatic event.

One embodiment provides a method including performing a process thereby to test and/or validate, via the second data set, a hypothesis as to the nature of a brain injury made based on the first data set.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

### BRIEF DESCRIPTION OF THE DRAWINGS

Embodiments of the invention will now be described, by way of example only, with reference to the accompanying drawings in which:

Embodiments of the invention will now be described, by way of example only, with reference to the accompanying drawings in which:

FIG. 1A to FIG. 1D illustrates an instrumented mouthguard in varying states of assembly.

FIGS. 2A and 2B illustrate an example PCB component for an instrumented mouthguard.

FIG. 3 illustrates a technology framework according to one embodiment.

FIG. 4A to FIG. 4C illustrate example use cases for technology described herein.

FIG. 5 illustrates a VR technology framework according to one embodiment.

FIG. 6 illustrates a plurality of test types according to one embodiment.

FIG. 7A to FIG. 7C illustrate example methods.

### DETAILED DESCRIPTION

The present invention relates, in various embodiments, to technology adapted for improved assessment of cognitive function in a human subject. This included, but is not limited to, assessment of cognitive function affected by brain injuries, for example injuries sustained during sporting activities. In some embodiments a virtual reality system is used to apply controlled cognitive loading to the subject, via a series of distinct test types which in combination apply an increasing cognitive load over time.

Embodiments described below focus primarily on assessment of actual/suspected trauma-related brain injuries, for example in the context of contact sports (for example various forms of football, MMA, boxing, action sports, and the like). However, it will be appreciated that various technologies described herein may be applied in other situations, for example in the context of determining impairment due to drugs, alcohol, disease, or other physiological factors.

In some embodiments, results are optionally assessed in conjunction with external data, relates to an underlying condition that may affect cognitive function, such as a brain injury or medical condition. In some cases this may include data that is collected during an impact event related to a suspected brain injury. For example, this may be data from a sensor device such as an instrumented mouthguard device, and/or data from Finite Element Analysis (FEA) model (which may be fed by data from a sensor device such as an instrumented mouthguard device). In the context of medical conditions, the external data may include pathology, body/brain scans, and/or other measures of human biological condition. While some embodiments will be described herein with particular reference to the use of external data, it will be appreciated that the invention is not limited to such a use case, and is applicable in broader contexts.

### Subject Assessment Via Cognitive Load Control

Some embodiments relate to assessment of a cognitive function (for example brain injury or other physiological condition) via delivery of tests using virtual reality hardware.

As used herein, the term “virtual reality hardware” (VR hardware) is used to describe a wearable computer device which includes a display screen and motion sensors such as Inertial Monitoring Units (IMUs), which enable a user to observe a virtual three-dimensional space via head movements. The virtual reality hardware also includes one or more input devices, for example handheld controllers, triggers, buttons, microphones, and the like. In some cases additional peripheral stimuli devices may be included, for example a balance board device that is configured to deliver stimuli to upset a user’s balance (relevant for vestibular system testing). In some embodiments VR hardware is controlled via a connected computer system, including one or more local and/or networked computer systems. These are used to control delivery of tests via the VR system, and for the collection and/or analysis of test results.

It is assumed for the purposes described herein that the VR hardware does not provide eye tracking functionality. Eye tracking functionality is used by a some known cognitive assessment technology platforms. However, such technology is complex, expensive, and often unreliable. As such, technology described herein has been adapted to operate without a need for eye tracking. In further embodiments the present technology may optionally be combined into a technology system that provides eye tracking.

In overview, a premise of technology described herein (at least in some embodiments) is that assessment of neurological condition can be enhanced by delivering a sequence of tests of different test classes. By delivering such a sequence, and withing that sequence transitioning between different test of different classes, the sequence causes an increase in cognitive loading to a subject. Variations in performance of tests in particular classes observed as a result of this increase in cognitive loading is used to assess cognitive function and provide an indication of potential brain injuries.

In terms of tests that are applied, the technology makes use of a plurality of neurological tests that are renderable via hardware including a virtual reality system, with the plurality of neurological tests includes neurological tests belonging to a plurality of distinct test classes. In a preferred embodiment the test classes include a combination of the following:
Short-term memory tests.
Long-term memory tests.
Vestibular system tests. In some embodiments these make use of a peripheral balance board which is configured to measure variations of subject weighting in forward/backward/left/right directions in response to stimuli delivered via the VR hardware screen which is intended to upset the subjects balance For example
Reaction time tests. These may include ocular reaction time tests, whereby a user is presented with a visual stimulus and instructed to interact with an input device upon identifying that visual stimulus.
Executive cognitive function tests.

In some embodiments, a neurological assessment includes presenting a sequence of tests belonging to different classes (with adjacent tests having distinct classes) in a cyclical manner, and identifying variations in performance attributable to increasing of cognitive loading. This optionally includes comparing subject performance with a plurality of tests belonging to a particular one of the distinct test classes which are delivered non-adjacently with respect to the sequence.

### Example VR Testing Framework

FIG. 5 illustrates a technology framework configured to enable assessment of a brain injury or other physiological condition. For example, in some embodiments this technology framework is used as a means to assess brain injuries for participants in a content sport, for example as a “sideline” assessment tool (although described as “sideline”, it will be appreciated that the assessment would usually be administered in an indoor space proximal a sporting field).

The framework of FIG. 5 is configured to perform a neurological assessment of a human subject 500. Subject 500 wears a VR headset 501, which is optionally a commercially available “off the shelf” system (for example an Oculus Rift/Quest/Go, HTC Vive, PlayStation VR, or the like; or alternately a VR system which makes use of a smartphone or the like in a specialised housing). Headset 501 includes one or more Inertial Measurement Units (IMUs) 507, thereby to enable observation of movement and control over scene rendering in response, based on operation of a scene rendering module 505 which causes rendering of a 3D VR scene on an electronic display 504. Input/output modules 506 are configured to deliver additional output stimuli to subject 500 (for example auditory, haptic, lights, and so on) and receive inputs (for example input via a handheld input device 502).

VR Headset 501 is coupled to a computer system 510, which executes computer code via one or more processors thereby to control operation of headset 510. In some embodiments some or all functions of computer system 510 are embedded into the headset data.

System 510 (and other components in FIG. 5) is described by reference to various modules. The term “module” refers to a software component that is logically separable (a computer program), or a hardware component. The module of the embodiment refers to not only a module in the computer program but also a module in a hardware configuration. The discussion of the embodiment also serves as the discussion of computer programs for causing the modules to function (including a program that causes a computer to execute each step, a program that causes the computer to function as means, and a program that causes the computer to implement each function), and as the discussion of a system and a method. For convenience of explanation, the phrases “stores information,” “causes information to be stored,” and other phrases equivalent thereto are used. If the embodiment is a computer program, these phrases are intended to express “causes a memory device to store information” or “controls a memory device to cause the memory device to store information.” The modules may correspond to the functions in a one-to-one correspondence. In a software implementation, one module may form one program or multiple modules may form one program. One module may form multiple programs. Multiple modules may be executed by a single computer. A single module may be executed by multiple computers in a distributed environment or a parallel environment. One module may include another module. In the discussion that follows, the term “connection” refers to not only a physical connection but also a logical connection (such as an exchange of data, instructions, and data reference relationship). The term “predetermined” means that something is decided in advance of a process of interest. The term “predetermined” is thus intended to refer to something that is decided in advance of a process of interest in the embodiment. Even after a process in the embodiment has started, the term “predetermined” refers to something that is decided in advance of a process of interest depending on a condition or a status of the embodiment at the present point of time or depending on a condition or status heretofore continuing down to the present point of time. If “predetermined values” are plural, the predetermined values may be different from each other, or two or more of the predetermined values (including all the values) may be equal to each other. A statement that “if A, B is to be performed” is intended to mean “that it is determined whether something is A, and that if something is determined as A, an action B is to be carried out”. The statement becomes meaningless if the determination as to whether something is A is not performed.

The term “system” refers to an arrangement where multiple computers, hardware configurations, and devices are interconnected via a communication network (including a one-to-one communication connection). The term “system”, and the term “device”, also refer to an arrangement that includes a single computer, a hardware configuration, and a device. The system does not include a social system that is a social “arrangement” formulated by humans.

At each process performed by a module, or at one of the processes performed by a module, information as a process target is read from a memory device, the information is then processed, and the process results are written onto the memory device. A description related to the reading of the information from the memory device prior to the process and the writing of the processed information onto the memory device subsequent to the process may be omitted as appropriate. The memory devices may include a hard disk, a random-access memory (RAM), an external storage medium, a memory device connected via a communication network, and a ledger within a CPU (Central Processing Unit).

System 511 includes a virtual reality engine 511, which is configured to process predefined VR content and cause that to be rendered via scene rendering module 505. In the present embodiment, the VR content includes a series of interactive tests provided in VR test data 512. Data 512 includes code for enabling execution of a plurality of neurological tests, including neurological tests belonging to a plurality of distinct test classes. Additional detail regarding these tests and test classes is provided further below. A cognitive assessment control module 513 is configured to enable selection and execution of the tests, for example based on a one-by-one test selection, or via generation of a predefined playlist of tests.

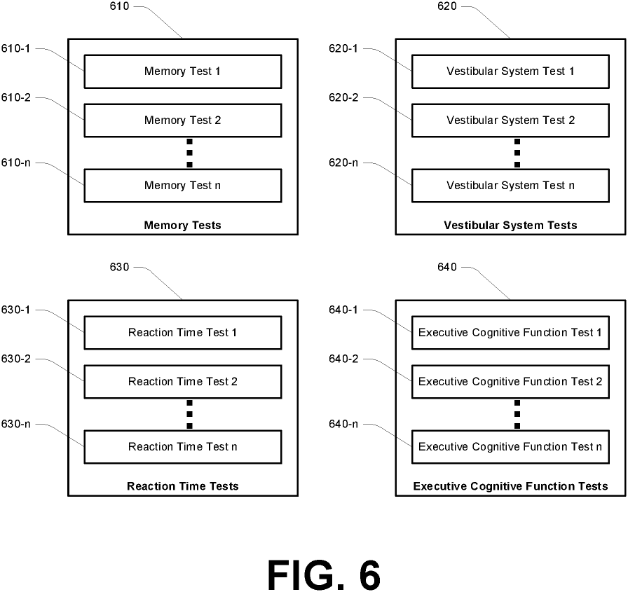

FIG. 6 illustrates an example of test data. This shows a plurality of test classes, being a class of memory tests 600, a class of vestibular system tests 620, a class of reaction time tests 630, and a class of executive cognitive function tests 640. Each class of tests includes a plurality of individual tests. In some embodiments one or more of the calluses incudes only a single test. Tests may include tests having one or more of the following properties:
Predefined stimuli which are displayed in a predefined sequence.
Stimuli which are presented in a randomised/partially randomised sequence.
Adjustable time parameters, for example stimuli presentation time and/or test total time.
Adjustable difficulty parameters, for example from “easy” to “difficult”.

In the illustrated example, system 511 also includes a balance board module 514, which is configured to interact with an electronic balance board 503. For example, a Balance Board such as that provided by Nintendo’s Wii Fit Plus may be used. Balance board 503 provides a signal representative of 2-dimensional weight distribution of subject 500, thereby to enable assessment of vestibular system responses to stimuli. In some embodiments board 503 is coupled to headset 501 rather than to system 511.

A cognitive assessment control system 520 is configured to operate in conjunction with system 510 for the purposes of processing results representative of subject 500’s performance in a neurological assessment, and in some embodiments for facilitating authoring/configuration of neurological assessments. In some embodiments systems 510 and 520 are defined by common computer hardware.

System 521 includes a cognitive assessment design module 521. Module 521 is configured to enable user authoring of neurological assessments, with each neurological assessment including instructions for causing sequential rendering of a sequence of neurological tests constructed from a subset of the plurality of neurological tests in data 512. This sequence of the neurological tests is preferably defined thereby to sequentially provide tests belonging to different ones of the plurality of distinct test classes, and in doing so thereby to deliver an increasing cognitive load. Module 521 preferably provides a user interface for facilitating the authoring; this may take the form of a playlist generator. Tools for authoring tests optionally include any one or more of the following:
A playlist authoring tool for defining a sequence of predefined tests.
A playlist authoring tool for defining a sequence of predefined tests, along with test parameters for each test. The test parameters may include, for example: a test duration and/or a test difficulty.
A rules editor configured to enable defining of rules for selection of a next test (and optionally parameters for that test) based on performance in one or more preceding tests.
A rules editor configured to enable defining of rules for selection of a test sequence based on input from a physical event data module 523, which receives data representative of a physical event preceding the assessment (for example data from an instrumented mouthguard or other wearable device, and/or from a FEA brain model that is executed based on data representative of a preceding physical event). For example, this allows for selection of a test based on an automated assessment of severity (for example severity of an impact or other traumatic event), affected brain region(s), and the like.
A rules editor configured to enable defining of rules for selection of a test sequence based on subject demographic data.
A rules editor configured to enable defining of rules for selection of a test sequence based on historical data for a user and/or user demographics, for example as stored in a user/benchmarking module 524. This optionally enables selection of assessments corresponding to those delivered to a particular user in the past, thereby to assist in comparative performance benchmarking, and/or selection of a test based on benchmarking data for users fitting specific demographic profiles for which benchmarking data is available.

A results processing module 522 is configured to receive and process results, in the form of subject performance data, derived from user interaction with the neurological assessment, thereby to enable assessment of neurological factors. This preferably includes identifying variations in performance attributable to increasing of cognitive loading resulting from transitioning between tests of different classes.

In use, cognitive assessment control module 513 is configured to deliver, to a subject using the virtual reality system, a neurological assessment including a sequence constructed from a subset of the plurality of neurological tests, wherein the sequence of the neurological tests is defined thereby to sequentially provide tests belonging to different ones of the plurality of distinct test classes, thereby to deliver an increasing cognitive load;. This results in generation of subject performance data representative of performance of the subject in the neurological assessment. The subject performance data is then processed thereby to derive one or more measures representative of subject neurological conditions.

In some embodiments, identifying variations in performance attributable to increasing of cognitive loading includes comparing subject performance with a plurality of tests belonging to a particular one of the distinct test classes which are delivered non-adjacently with respect to the sequence. For example, this may include comparing subject performance with a first test belonging to a particular one of the distinct test classes with a second test belonging to the same particular one of the distinct test classes, wherein the second test is delivered subsequent to the first test non-adjacently with respect to the sequence.

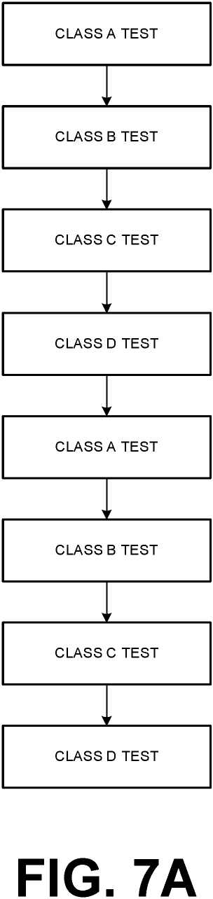

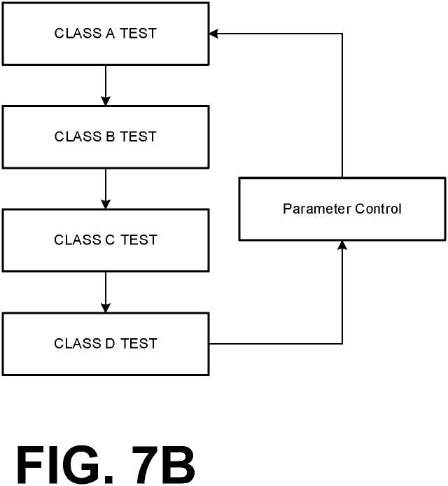

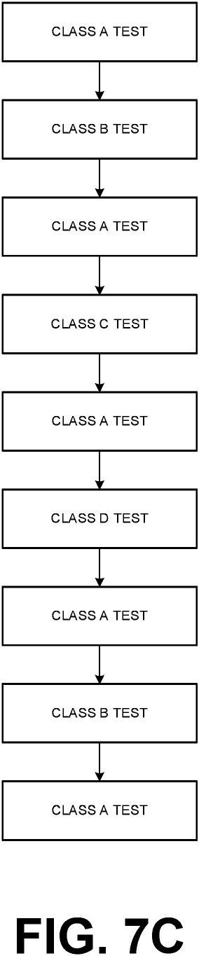

By way of example, FIG. 7A to FIG. 7C illustrate example sequences of tests which may make up all or part of a sequence defining a neurological assessment. These refer to a CLASS A; CLASS B; CLASS C and CLASS D, which may be classes 600-640 of FIG. 5. Referring to these examples:
In FIG. 7A, tests are delivered in a defined sequence in a cyclical manner, using the same test parameter each time (the sequence may be looped). This allows for a cognitive load to be increased and like-for-like performance testing to be performed on each cycle.
In FIG. 7B, a cycle is repeated with a parameter section process on each loop, thereby to enable variation of test parameters (for example duration and/or difficulty adjustment). This is optionally used to customise the rate at which a neurological load is increased. For example, relatively shorter test durations can be used to increase the rate of cognitive loading.
In FIG. 7C, a given class of test is repeated between tests of cycling classes. This allows for regular benchmarking of performance during cognitive loading. In some embodiments the repeated class is selected based on a prediction of impairment derived from a FEA model of the main used to assess a particular preceding traumatic event.

It will be appreciated that these are examples only, and other test sequencing approaches may be used in further embodiments.

### Example Testing Protocol

An example testing protocol is described below. It should be appreciated that this is an example only, and that various modifications can be made whilst remaining within the scope of the present invention.

The example testing protocol includes three high level categories:
Memory. This includes a test type for each of immediate memory, a test for working memory, and a test for delayed memory.
Oculomotor behaviour. This includes a visual assessment test, and a visual reaction test.
Vestibular. This includes a further visual reaction test, an acoustic reaction speed test, and a motor system posture control test.

Context to these tests, and examples of how the tests are implemented via the VR system are described in the following sections.

### Memory Testing - Context

One of the most common symptoms after concussion are memory impairments and attention deficits (Chen et al. 2004; McAllister et al. 1999, 2006). Even in asymptomatic patients, memory deficits can be found and are indicative of a concussion (Broglio et al. 2007).

Although in most patient these impairments resolve within the first few days after injury incident, in some cases they can become a long-term problem (Belanger et al. 2010; Gardner et al. 2010; Lovell et al. 2003; Reddy & Collins 2009). There is increasing evidence, that exposure to recurrent head impacts and concussions increases the likelihood for persistent memory impairments and other cognitive deficits (Amen et al. 2016; de Beaumont et al. 2009; Hume et al. 2017; Koerte et al. 2016b,a; Stamm et al. 2015; Wilde et al. 2016; Wright et al. 2016).

Many neuroimaging studies have been performed with the sophisticated aim to identify structural abnormalities that might explain chronic memory deficits. Cortical thinning is observed in retired professional athletes with memory dysfunctions and history of concussions (Koerte et al. 2016b). Frequently, changes in white matter are associated with memory impairments and reduced processing speed in concussed professional athletes (Bazarian et al. 2012; Wilde et al. 2016). There is some evidence, that often damaged brain regions in NFL players experiencing memory loss show abnormally low blood flow (Amen et al. 2016). Hippocampal atrophy and volume loss have been observed in TBI and are correlated with cognitive impairments including memory (Himanen et al. 2005; Strain et al. 2015).

Some studies aim to correlate abnormalities in brain activation pattern to memory impairments (Functional et al. 2013). It has been shown that that activation pattern of working memory in mild TBI patients differs from the control group (McAllister et al. 1999). Another interesting study found that those memory related activation pattern became comparable to those observed in control groups once symptoms have been resolved (Chen et al. 2004).

Despite abundance studies the mechanisms and factors contributing to transient and chronic memory impairments are poorly understood.

To improve current assessment methods by deciphering the effects of concussion on memory, the first step is understanding the mechanisms of memory formation and maintenance. There have been several models suggested to describe memory; however, the most influential model suggested over 50 years ago by Atkinson and Shiffrin is the multi-store or modal model (Atkinson & Shiffrin 1968). This model proposes that human memory has three separate components, namely the sensory memory, the short-term memory, and the long-term memory. All information we receive -consciously or subconsciously- enters our awareness through the sensory systems, e.g. visual or auditory system, and stays there for a short time of several hundred milliseconds. Once we pay attention, we can store this information for a short period of several seconds in our short-term memory. After roughly 15 to 30 seconds the information is forgotten through decay or displacement unless we can keep maintaining it actively through recall. Eventually, repeated rehearsal can transfer this information to the long-term memory, where we can retrieval the information after days, months and even years.

Later in 1974, Baddeley’s model of working memory attempts to describe short-term memory more accurately by subdividing it into three further components (Baddeley & Hitch 1974). The Central executive is supervising the information flow from and to its two slave systems, namely the Visuospatial sketchpad, and the Phonological loop.

The Central executive acts as a supervisory system through directing focus and target information, this way making sure that short-term memory is actively working and can interact with long-term memory. It encodes, updates, and deletes information, structures information, controls attention and changes strategies in a task bound manner. This way, the Central executive controls the flow of information from and to its two lower systems, the Phonological loop, and the Visuo-spatial sketchpad.

The phonological loop (or “articulatory loop”) deals with sound or phonological information. This means even visually presented information can be articulated silently and encoded into the phonological storage. The visuo-spatial sketchpad keeps visual information, enables to create, revisit, and manipulate mental images.

As we can see in these very simplified memory models, memory formation is a process that depends on several factors including sensory input and attention. Therefore, while finding the cause of memory deficits can be challenging, the assessment of memory is highly sensitive to cognitive misfunctioning. Because of the complexity of factors contributing to and divers processing pathways involved in memory formation, it is likely that cognitive impairments are detected by memory tests. For instance, if an athlete has attention deficits and cannot take up new information - maybe because of slowed down processing times- it is logical that his short-term memory performance shows deficits.

### Memory Testing - Example Testing Protocol

Memory aspects of the example testing protocol are configured to test three aspects of memory, the immediate memory, working memory and delayed memory. The tests are described as Memory Test 1, Memory Test 2, Memory Test 3, and Memory Test 4.

In a Memory Test 1, the user interface the VR system is controlled to present a list of words for a defined period of time. Next, the user interface is controlled such that the participant is exposed to listed or decoy words one by one, and asked whether that word was present in the original list.

In Memory Test 2, the presentation of listed and decoy words is repeated (with the same list). This two tasks, where short-term memory is tested within the range of seconds to minutes, induce a learning process: The participant has time to take up information and through rehearsal memory is trained.

In Memory Test 3, which is delivered by the user interface of the VR system after performing the rest of the assessments (oculomotor and vestibular), the delayed memory is tested by recalling the same word list as per Memory Test 1 and Memory Test 2, without repeated exposure to the list (again optionally using listed/decoy word classification as a testing means).

Memory Test 4 aims to test the working memory. Five to nine nodes are shown, with one saying ‘START’ and another one saying ‘END’. A pattern connecting the nodes is drawn by the computer from start to end and disappears after a few seconds and the participant has to redraw the pattern. To ensure that the participant is not guessing (as opposed to utilising working memory), each pattern task is ended as soon as a wrong node is connected.

### Oculomotor Behaviour Testing - Context

Assessment of oculomotor behaviour plays a major role in diagnosing neurological abnormalities because of its complex neurocircuitry; functional neuroimaging has shown association of neuronal dysfunction with oculomotor performance (Bedell and Stevenson 2013; Johnson, Zhang, et al. 2015).

Oculomotor behaviour is divided into three eye movement categories: fixation, smooth pursuit, and saccades where we distinguish between vertical and horizontal saccades (Land and Tatler 2012). For fixation, the eye position is kept relatively still to focus a stationary image on the fovea, the central area on the retina with the highest sharp vision. Smooth pursuit is the targeting process of a moving stimulus to stabilize the image on the fovea. The saccades are rapid reflex-like eye movements between at least two fixation points. The different types of eye movement are associated with activation of different brain region, e.g., certain cortical areas control timing and location of saccades while cerebellar structures regulate saccade size and accuracy (Wong 2008). Further, stimuli direction activates different brain regions for saccades: For instance, horizontal saccades are initiated by the paramedian pontine reticular formation in the pons that receives inputs from the frontal eye field (FEF). For vertical saccades, the FEF signals to the rostral interstitial nucleus of the medial longitudinal fasciculus in the midbrain.

Saccades are usually characterized by velocity, duration, accuracy, and initiation time. We can test voluntary or self-paced saccades, i.e., saccades made between two stationary stimuli in a fixed time window. Traumatic brain injuries including concussion can affect eye movement like saccadic behaviour in humans. In individual TBI patients, the saccadic impairments have been associated with the degree of their head injury (Ventura et al. 2015, 2016).

In concussion assessment, balance and cognitive abilities are tested, without exploiting the potential of eye movement assessment: Common tests such as SCAT5 ask about the ability of the patient to open their eyes and blurred vision while visual deficits are not assessed in detail. Symptom reports are unreliable, subjective, and quick cognitive assessments alone as memory tests in SCAT5 can fail to detect a range of cognitive deficits. Therefore, it is recommended in several studies to include assessment of eye movements in concussion assessment, e.g., via the King-Devick (K-D) test, which is a rapid visual performance measure (Mucha et al. 2014). It examines reading speed and language production and has a short assessment of saccade but does not test other eye movements. There are other tests like Vestibular/Ocular Motor Screening (VOMS) assessing amongst other saccades, smooth pursuits, and fixations (Ventura, Balcer, and Galetta 2014). However, the VOMS underlies subjective human bias and has not come into general use in sport-related concussion assessment yet. Emphasizing that vision uses half the pathways in the brain, it has great potential for concussion assessment (Heitger 2003; Heitger et al. 2009; Ventura et al. 2014). Roughly 65-90% of concussed patients reveal oculomotor disruptions, slowed saccades and deficits in smooth pursuit (Cochrane et al. 2019). A study in 2019 showed that saccadic and visual reaction times are significantly lower in concussed individuals (Hunfalvay et al. 2019, 2020).

Furthermore, recent studies showed that horizontal and vertical saccades can be used as diagnostic marker for TBI (Cochrane et al. 2019; Heick and Bay 2021; Hunfalvay et al. 2019, 2020; Stuart et al. 2020). The saccadic velocity, accuracy and targeting was measured in healthy controls and TBI patients of different TBI severity. The performance on targeting alone could distinguish between patients with mild and severe TBI and healthy controls. In alignment with previous studies, it was shown that horizontal saccades and targeting are most sensitive to distinguish between the different test groups.

With progressing technology, many devices, that can track eye movements, and several evaluation metrices have been developed (Cochrane et al. 2019). However, the influence of cognitive and visual functions on eye-movement has not been considered. Moreover, the validity and reliability of those tools and the processing of eye tracking data is insufficiently reported. The analysis methods as the tools are yet in a development stage. One study even reports that tests were repeated until a valid trial was completed (Andersson et al. 2010).

A good overview over the many different available eye movement instruments is provided in a critical review by Stuart et al. (2020) (see table below). Most studies used infrared eye tracking devices in a seated position, a few while standing or walking. Sampling frequencies of 50 - 200 Hz were reported for saccadic eye movement detection, while sampling rates of 60 Hz are sufficient to detect saccades (DiCesare et al. 2017; Johnson, Zhang, et al. 2015; Johnson, Hallett, and SlobouNovember 2015), a higher frequency should be considered to detect all features or deficiencies of eye movements (DiCesare et al. 2017; Johnson, Zhang, et al. 2015; Johnson, Hallett, et al. 2015). Another weakness of those eye movement instruments that needs to be considered is the vulnerability of the technology to head and body movements and calibration errors.

Optimization of the tools (reliability and validity) and standardization of analysis metrices needs to be further progressed until eye-tracking devices can be used as standard concussion assessment aids.

### Oculomotor Behaviour Testing - Example Testing Protocol

Common (computerized) neuropsychological test batteries focus on cognitive aspects and do not include assessment of vision and saccades. For the present of the example testing protocol described herein, we include additionally to the cognitive performance a hand-eye coordination test and a vision test that challenges the horizontal smooth pursuit (in a preferred embodiment taking in 2 minutes).

The example testing protocol includes a test (OBT Test 1) which challenges those eye movements identified to be significantly affected in concussion without a need for eye tracking hardware. A discussed above, horizontal saccades, smooth pursuit and targeting are most sensitive to distinguish between controls and concussed patients. In the example testing protocol, a visual assessment test is delivered via the VR hardware in which:
The subject is presented with a graphic showing a ball bouncing between two walls.
The subject is instructed to track the ball only by moving eyes with a fixed had position.
The subject is instructed to provide a designated input every time the ball hits the wall.

The software logic underlying the test is configured to cause the difficulty slowly over the two minutes (e.g. by increasing ball velocity and changes in direction). Changes in velocity and direction are randomised to challenge the subject’s predicting abilities and attention. Performance is evaluated by the accuracy of task performance. In case that a subject experiences abnormality in the oculomotor functioning, such as previously mentioned slowed saccades, troubles targeting for smooth pursuit, this is reflected in overall performance metrics (i.e. accuracy in providing inputs).

The example testing protocol also includes a test (OBT Test 2) which assesses hand-eye coordination and visual reaction, amongst other factors: The subject is presented with a virtual environment in which a game is presented. The game includes objects being thrown (or otherwise travelling through the air) towards the subject. The subject is instructed to catch the objects. Here, increase the difficulty levels is achieved by changes in ball velocity and number of balls in the game. The test is administered with the subject in a seated position.

### Vestibular Testing - Context

Humans have a set of vestibular organs on each side of the head, directly behind and interconnected to the acoustic system. The vestibular system detects the position and movement of the head and contributes this way most significantly to the sense of balance. It helps to coordinate movements of head, eyes, and our body posture. This coordination is an automated process that of which a person is unaware. However, if this system is malfunctioning, it can lead to many different symptoms such as sickness, motions sickness, vertigo, dizziness, nausea, and uncontrollable eye movements, which are all commonly reported symptoms after a concussion (Bear, Connors, and Paradiso n.d.; Calzolari et al. 2021; Echemendia et al. 2017).

The vestibular system is interconnected with several functions, e.g., our sight including visual processing pathways. Thus, damage to a broad range of brain areas such as the inner ear, nerve, brainstem, cerebellum, and cerebral hemispheres could all affect vestibular functioning. Therefor a multi-level assessment of balance and vestibular related eye-movements is required to screed those functions.

The vestibular system, like the acoustic system the cochlea, uses hair cells to translate movements. These hair cells are in interconnected in fluid filled chambers, the vestibular labyrinth. It consists of two structures, the otolith organs and the semicircular canals. The former detects the force of gravity and acceleration such as tilts of head. This way, we naturally sense where “up” and “down” is. The semicircular canals sense head rotations and angular accelerations. These canals are orientated in three directions, in approximately orthogonal planes i.e., 90 ° between any two of them. By this means, they can detect different kind of movement and acceleration, each in a different direction.

The vestibular system does not only help to control head, eye, and body position with the information about gravity and acceleration, it uses at the same time feedback from other body parts to finetune this information. The vestibular system receives itself inputs from the brain including the cerebellum (part of hindbrain) like visual, acoustic and the motor system, which all contribute to our sense of balance to maintain posture. For example, it is interconnected with spinal motor neurons, that control the leg muscles. Hereby, we can maintain balance even on a dynamic surface or object, such as on a surfboard. Another important function of the vestibular system is to maintain our visual focus on a moving point or when the body is moving, called the vestibulo- ocular reflex. This reflex triggered system can directly control the eye muscles meaning the vestibular system is connected to our oculomotor functions.

The assessment of balance in TBI and concussion is a relatively important factor for return to play decisions (link to intro chapter). In some cases, patients even develop chronic imbalance problems (Hoffer, Balough, and Gottshall 2007). A recent study indicated that 62% of TBI patients show dysfunctional balance performance while half of them did not report any balance problems which indicates the necessity of balance assessment in concussion management (Marcus et al. 2019). The mechanisms behind balance dysfunction and vestibular function in patients with TBI is not yet well understood. Concussion studies addressing this question are still evolving (Calzolari et al. 2021).

There is evidence that the cause for imbalance in a majority of acute TBI patients lies in vestibular related dysfunctions (Marcus et al. 2019; Sargeant et al. 2018). Interestingly, patients with dysfunctional vestibular system, perform better walking than standing (Brandt, Strupp, and Benson 1999; Calzolari et al. 2021).

### Vestibular- Example Testing Protocol

In the example testing protocol, tests are implemented to cover various different functions that contribute to the sense of balance. Apart from the vestibular system, balance depends on visual and acoustic cues as mentioned above. In addition, it has been shown that dual tasks in balance are more effective to detect balance impairments (Beauchet and Berrut 2006; Camicioli et al. 1997; Howell, Buckley, et al. 2018; Howell, Kirkwood, et al. 2018).

In view is this, the sample testing protocol has three tests (Vestibular Test 1, Vestibular Test 2 and Vestibular Test 3) performed in a standing position on the balance board. The system is configured to collect data about postural stability during different conditions and tasks.

Vestibular Test 1 is a repeat of OBT Test 1 (or optionally OBT Test 2), but in a standing position. Gaze control and saccades are less accurate in a standing position than sitting position (Boulanger, Giraudet, and Faubert 2017; Rougier and Garin 2007). By this approach, the system is able to determine whether standing affects results of the visual reaction test.

Vestibular Test 2 relates to acoustic reaction speed. The vestibular and auditory system are connected at an early processing stage because the organism is exposed constantly to moving sound sources from the environment while moving itself. Auditory inputs can improve postural stability, these two senses influence each other. The dorsal cochlear nucleus where auditory nerve fibres form their first synapses, integrates auditory and vestibular information (Rougier and Garin 2007; Stevens et al. 2017; Wigderson, Nelken, and Yarom 2016). Interestingly, patients with vertigo compensate the feeling of imbalance by focusing more on visual cues and are highly visually dependent (Bronstein 1995). Surprisingly, they show better adaptation to visual disorientations (Guerraz et al. 2001). Because our vision too contributes to postural stability, and there is a slight chance that patients can adapt to the feeling of imbalance by stronger visual dependency, the auditory reaction test is performed in a dark room in the VR environment (Boulanger et al. 2017; Rougier and Garin 2007).

In Vestibular Test 3, the balance board itself is used here as a controller. This task is performed by instructing the subject to engage in a process of shifting weight from one foot to the other, forward, and backward, i.e., the centre of pressure or the centre of gravity on the board is moved. This aims to challenge control of the motor system that controls posture, and provides a test that required precise control of posture:

### Integration With Instrumented Mouthguard Technology

In some embodiments, VR technology is used to assess cognitive function in combination with data derived from observing a traumatic event (such as a head impact). This observation may be achieved via a worn device having motion sensors, for example an instrumented mouthguard device. An example of an instrumented mouthguard device is provided further below.

One class of embodiment provides a method for assessing a brain injury, the method including: accessing a first data set representative of an observed traumatic event, wherein the first data set is generated in response to data derived from one or more subject-worn motion sensors; accessing a second data set representative of neurological conditions following the observed traumatic event, wherein the second data set is generated in response to data derived from subject performance data in a neurological assessment delivered by a virtual reality system; and processing a combination of data from the first data set and the second data set thereby to define a third data set representative of an enhanced brain injury assessment.

A second class of embodiment provides a method for assessing a brain injury, the method including: accessing a first data set representative of an observed traumatic event, wherein the first data set is generated in response to data derived from one or more subject-worn motion sensors; based on the first data set, configuring a virtual reality system to deliver a neurological assessment having defined parameters to the subject, and in response define a second data set representative of subject performance in the assessment; and performing a brain injury assessment based on a combination of the first data set and the second data set.

In this manner, data relating to a actual physical traumatic event may be used for either or both of: (i) influencing parameters of a VR-based cognitive assessment which is delivered to a subject; and (ii) providing an enhanced assessment which combines data derived from observation of the event with results of a VR neurological assessment.

The first data set in some embodiments includes a metric derived from processing of data provided by the instrumented mouthguard device (for example a metric representing severity of trauma, preferably derived from measurement of rotational acceleration of the subject’s head). In other embodiments the first data set includes output from a brain model that is executed base on the data derived from an instrumented mouthguard device (or another device providing one or more subject-worn motion sensors). This may include a brain model in the form of a Finite Element Analysis (FEA) model which makes use of head motion data (for example linear and/or rotational accelerations) thereby to model predicted effects on the internal structure of the brain.

In some embodiments, a processing method includes processing a combination of data from the first data set and the second data set by identifying a correlation between an output of the FEA model and performance in the neurological assessment. For example, this may include benchmarking against prior results for the same subject and/or different subjects. This optionally enables testing predictions of injury severity and effect on subject function, leading to a better understanding of the effects of a particular traumatic event.

In embodiments where VR assessment derived data and trauma-derived data are combined, a process is optionally performed thereby to derive metric representative of a deviation between: (i) expected performance in the neurological assessment based on the observed traumatic event; and (ii) actual performance in the neurological assessment based on the observed traumatic event. This is optionally used to test and/or validate, via the second data set, a hypothesis as to the nature of a brain injury made based on the first data set.

### Instrumented Technology Overview

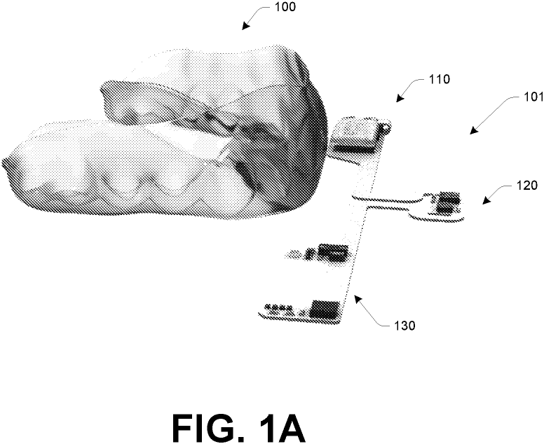

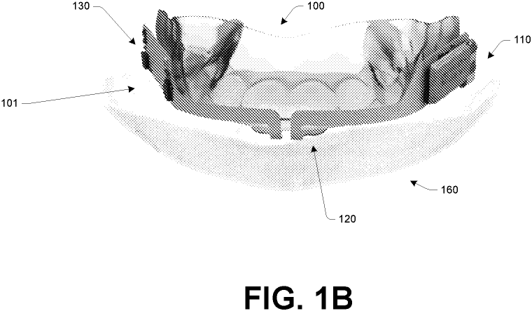

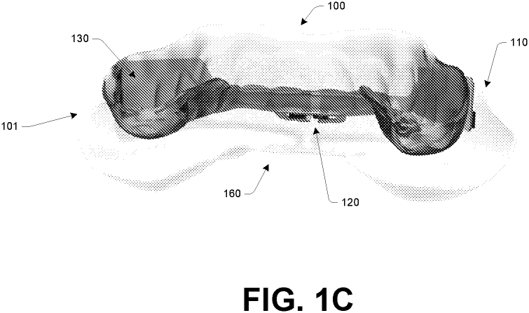

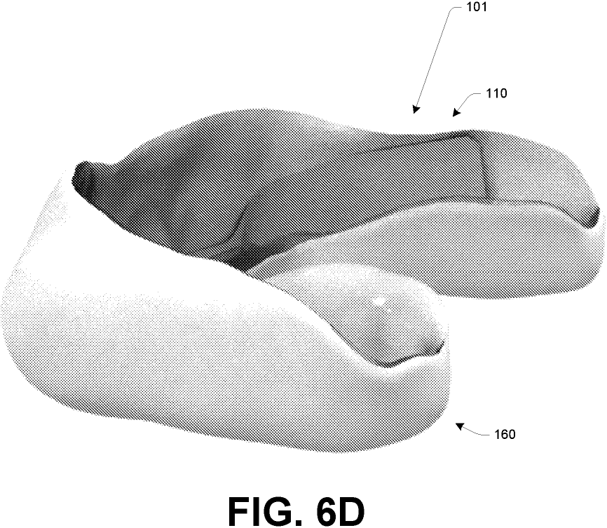

FIG. 1A to FIG. 1D illustrate an instrumented mouthguard device according to one embodiment. This example instrumented mouthguard is configurable to operate as a Head Impact Detection (HID) device, to provide both impact detection functionality and physical performance functionality.

The mouthguard comprises a mouthguard inner body 100, an instrumented component 101, and an outer mouthguard body 160. In the present embodiment the mouthguard inner body is custom formed based for a user based on a dentition scanning process, such that the mouthguard inner body provides a customised specifically to that user. The instrumented component 101 is then affixed to the inner body, and the outer body 160 sealed to the inner body 100 thereby to sandwich the instrumented component.

Additional detail regarding example instrumented mouthguard construction processes are provided in Australian Provisional Patent Application 2020904214, entitled “multilayered instrumented mouthguard devices, and methods for manufacturing of instrumented mouthguard devices”. The disclosure of that application is hereby incorporated by cross reference.

Instrumented component 101 includes a plurality of component zones 110, 120 and 130, which are spaced apart on a flexible PCB which follows a meandering path (i.e. the distance between component zones along the PCB is greater than the direct distance between the component zones).

The meandering path allows for mounting of the flexible circuit board substrate to the mouthguard inner body, such that the component zones are located in a frontal region of the mouthguard body (component zone 120); a side region of the mouthguard inner body (component zone 110); and an opposite side region of the mouthguard inner body from the second component zone (component zone 130). The frontal region is located on an opposite side of a teeth-receiving protective channel to the side region and opposite side region. In this example the frontal region is located on an inner side of the body relative to the protective channel, and the side region and opposite side regions are located on an outer side of the body relative to the protective channel. Outer body member cover 160 is mounted to the body thereby to seal components mounted on both the outer side of the inner body relative to the protective channel thereby to cover and the inner side of the inner body relative to the protective channel.

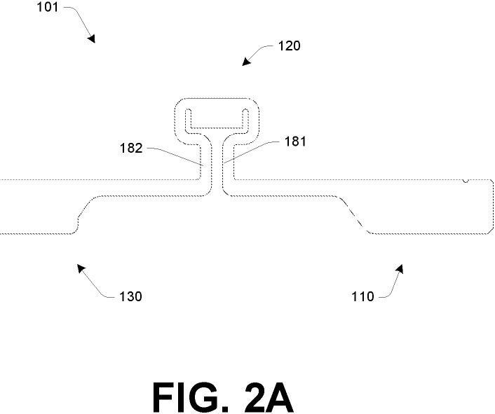

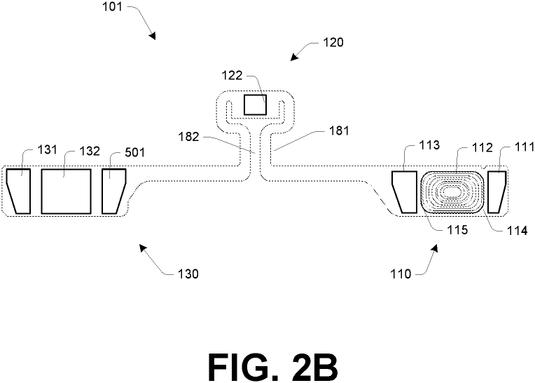

FIG. 2A and FIG. 2B illustrates an instrumented component 101 according to a further embodiment, this being configured for mounting in a mouthguard body thereby to provide an instrumented mouthguard.

As shown in FIG. 2A, component 101 is defined by a flexible circuit board substrate which is configured such that one or more conductive members electronically couples component zones (e.g. printed circuit board regions). The flexible circuit board in this manner defines a conductive member which is irregularly shaped such that it is configured to enable fitting of the component zones at desired locations on mouthguard bodies of varied shapes and sizes. More particularly, a PCB is formed to meander between component zones in a manner that allows for customisable fitting, whilst providing for added flexibility and robustness when the mouthguard is used. This presents a significant advantage over non-meandering PCBs, or the use of wires interconnecting distinct PCBs.

The PCB substrate illustrated in FIG. 2A may be of variable thickness, and/or have rigidity supports applied, thereby to adjust rigidity on a special basis thereby to protect PCB components as required for robustness.

Component 101 includes three component zones:
A right side component zone 110. In some implementations the right side component zone is configured to support PCB components including an accelerometer(3-axis), wireless communications unit, memory and microprocessor.
A frontal component zone 120. In some implementations, component zone 120 is split provides an accelerometer supporting zone configured to be positioned on the outer side of the front teeth (for a 3-axis accelerometer).
A left side component zone 130. In some implementations the left side component zone provides mounting locations for an accelerometer (3-axis), battery charging unit, and a battery mounting location.
The positioning of components described above, and shown in FIG. 2B, is an example only, and in other embodiments alternate configurations of components are distributed between the component zones.

A flexible connector member, defined by part of the PCB substrate onto which conductors connects these zones, has a first segment 181 which electronically couples right size component zone 110 and frontal component zone 120, and a second segment 182 which electronically couples front component zone 120 and left side component zone 130. As shown in FIGS. 2A and 2B, these segments are meandering. In this example, as with examples above, the meandering is such that, segment 181 is greater than the length of the separation of connection points with zones 110 and 120, and segment 182 is greater than the separation of connection points with zones 120 and 130.

The flexible connector member provides a flexible substrate onto which conductive strips and a plurality of PCB components are mounted (for example PCB components in zones 110, 120 and 130). In some embodiments the flexible substrate has an increased thickness in certain regions thereby to provide increased rigidity for PCB components that are susceptible to damage as a result of PCB flexion (for example see regions 111, 112 and 113 discussed below). In some embodiments additional materials are applied to the flexible substrate thereby to increase rigidity where required.

In the embodiment of FIG. 2B, zone 110 is defined by three substantially rigid PCB regions 111, 112 and 113, interconnected by comparatively flexible regions (flex connectors) 114 and 115. This enables a better fit of zone 110 to a curved surface; in the present embodiment it is configured to mounted in a right cheek region of the mouthguard body. Zone 110 includes a range of electronic components, including:
A 3-axis accelerometer.
A microprocessor (for example a Qualcomm CSR1012).
A memory module (for example a Macronix MX25L3233).
A wireless communications module, in this embodiment being a Bluetooth module coupled to a Bluetooth antenna (not shown), for example, an antenna configured to be mounted such that it runs across a frontal region of the mouthguard forward of a wearer’s teeth.
A coupling port to a programming tab (not shown).
A Light-Emitting Diode configured to be visi/ble through the mouthguard body (not shown), in order to provide a device state indication to a user. For example, this is configured to be positioned behind the wearer’s top lip.

It should be appreciated that the variations in rigidity within zone 110 (and across the component generally) is selected based at least in part of PCB components that are to be mounted at the various locations. For example, in one embodiment one or more of regions 111, 112 and 113 is not rigid, thereby to allow improved curvature upon application to the mouthguard body, and PCB components mounted to the non-rigid region are selected and/or mounted in such a manner to remain robust in spite to flexion in the PCB substrate.

Zone 120 includes a PCB region 122 including a 3-axis accelerometer (which is configured to be mounted to the mouthguard body in a location that in use is positioned behind front teeth).

Zone 130 is configured to be mounted on a left cheek region of the mouthguard body, and includes a PCB that carries a 3-axis accelerometer 131, along with a charging coil 132 to enable wireless charging of a battery unit 151.

In other implementations the battery unit is located in zone 110 or zone 120. In further embodiments additional components including the likes of gyroscopes may also be present at one or more of the component zones (for example a gyroscope in combination with an accelerometer at each component zone.

Segment 181 of the conductive member is configured such that, upon mounting to the mouthguard body, it traverses across a bottom region of the mouthguard body at a region approximately adjacent cuspid and first bicuspid (or, alternately, first and second teeth). This allows zone 120 to be provided on an internal region (behind teeth) and zone 110 provided on an external region (in front of teeth). A sealing cover is mounted to the body thereby to seal components mounted on both the outer side of the body relative to the protective channel thereby to cover and the inner side of the body relative to the protective channel.

In a further embodiment, component 101 or a variant thereof is embedded into a post-manufacture customised (e.g. a “boil and bite”) mouthguard. In such an embodiment, a standard generic form is injection moulded, and a user heats the mouthguard into a temporarily deformable state and bites firmly into it thereby to shape the resilient materials substantially to their teeth before it cools and becomes stable in the new customised shape.

### Example HID Technology Framework

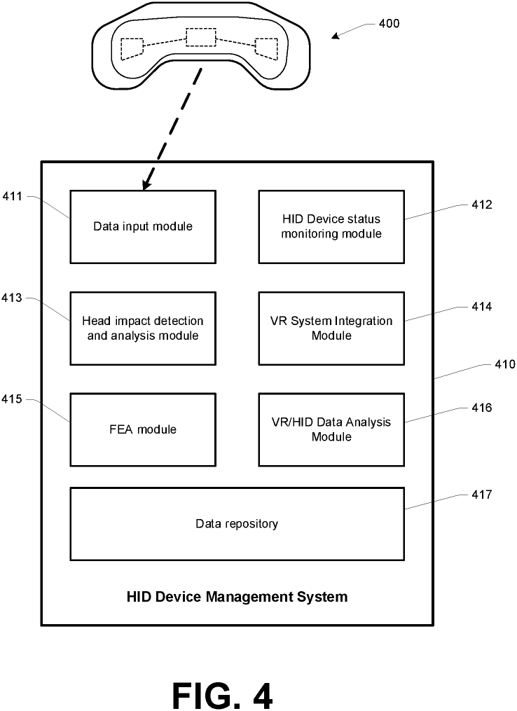

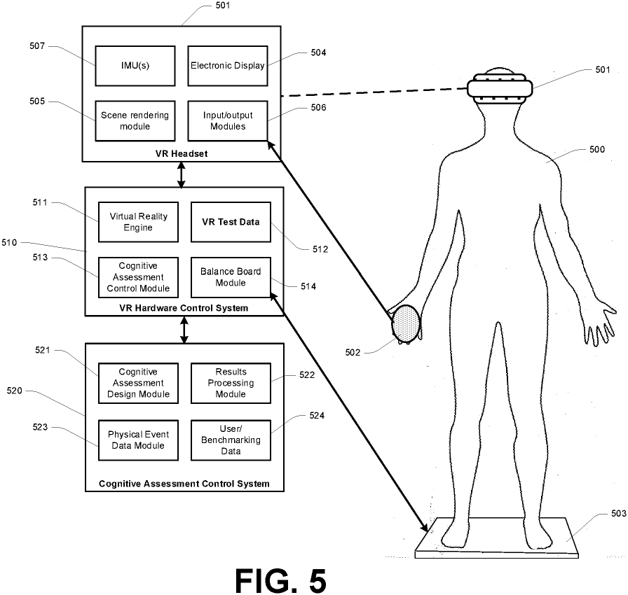

FIG. 4 illustrates an example HID technology framework, configured to enable monitoring of head impacts and physical performance for one or more subjects in a sporting activity.

The framework is described by reference to an HID device, in the form of a instrumented mouthguard 400, and an HID Device Management System 410, which takes the form of a computing device (for example a PC, notebook, tablet or smartphone) or a plurality of computing devices (for example various processing functionalities may be performed by cloud-hosted components). Instrumented mouthguard 400 includes a microprocessor configured to execute onboard software instructions, and it will be appreciated that various functions described as being performed by system 410 may in further embodiments be performed in whole or in part by mouthguard 400.

Software is described herein by reference to various modules. The term “module” refers to a software component that is logically separable (a computer program), or a hardware component. The module of the embodiment refers to not only a module in the computer program but also a module in a hardware configuration. The discussion of the embodiment also serves as the discussion of computer programs for causing the modules to function (including a program that causes a computer to execute each step, a program that causes the computer to function as means, and a program that causes the computer to implement each function), and as the discussion of a system and a method. For convenience of explanation, the phrases “stores information,” “causes information to be stored,” and other phrases equivalent thereto are used. If the embodiment is a computer program, these phrases are intended to express “causes a memory device to store information” or “controls a memory device to cause the memory device to store information.” The modules may correspond to the functions in a one-to-one correspondence. In a software implementation, one module may form one program or multiple modules may form one program. One module may form multiple programs. Multiple modules may be executed by a single computer. A single module may be executed by multiple computers in a distributed environment or a parallel environment. One module may include another module. In the discussion that follows, the term “connection” refers to not only a physical connection but also a logical connection (such as an exchange of data, instructions, and data reference relationship). The term “predetermined” means that something is decided in advance of a process of interest. The term “predetermined” is thus intended to refer to something that is decided in advance of a process of interest in the embodiment. Even after a process in the embodiment has started, the term “predetermined” refers to something that is decided in advance of a process of interest depending on a condition or a status of the embodiment at the present point of time or depending on a condition or status heretofore continuing down to the present point of time. If “predetermined values” are plural, the predetermined values may be different from each other, or two or more of the predetermined values (including all the values) may be equal to each other. A statement that “if A, B is to be performed” is intended to mean “that it is determined whether something is A, and that if something is determined as A, an action B is to be carried out”. The statement becomes meaningless if the determination as to whether something is A is not performed.

The term “system” refers to an arrangement where multiple computers, hardware configurations, and devices are interconnected via a communication network (including a one-to-one communication connection). The term “system”, and the term “device”, also refer to an arrangement that includes a single computer, a hardware configuration, and a device. The system does not include a social system that is a social “arrangement” formulated by humans.

At each process performed by a module, or at one of the processes performed by a module, information as a process target is read from a memory device, the information is then processed, and the process results are written onto the memory device. A description related to the reading of the information from the memory device prior to the process and the writing of the processed information onto the memory device subsequent to the process may be omitted as appropriate. The memory devices may include a hard disk, a random-access memory (RAM), an external storage medium, a memory device connected via a communication network, and a ledger within a CPU (Central Processing Unit).

In the example of FIG. 4, instrumented mouthguard 400 communicates with system 410 via a wireless connection. This may include a range of wireless technologies, including WiFi, Bluetooth, and/or other radio bands. In some embodiments communications between mouthguard 400 and system 410 progresses via one or more intermediate devices, including on-body retransmitting devices, devices in mesh networks, routers, and so on.

Data transmitted by mouthguard 400 is received by a data input module 411. Data input module 411 is configured to extract and sort input data, thereby to organise that data into memory accessible to system 410 (for example in one or more databases). This includes identifying a unique device identifier associated with mouthguard 400, which is preferably associated with a unique human subject. The data may include, for example, any one or more of: (i) a time-series of sensor readings, with associated time correlation data (such as a timestamp at the commencement of the series, and a known sampling rate); (ii) data packets representative of identified potential impact events (for example where the mouthguard is configured to operate in at least one setting where it transmits sensor data only where that sensor data has threshold values which indicate a potential impact); and (iii) output data from an onboard processing module (for example on onboard FEA module which provides an output based on a dosage input, the dosage input being derived from sensor data); and (iv) regular beacon/heartbeat data packets representative of device status. Other data may also be received, for example physiological data (such as heart rate, breathing rate, etc).

Data received and processed via input module 411 is stored in a data repository 417, where it is available for accessing and processing by other modules of system 410.

A HID device status monitoring module 412 is configured to process data received via input module 411 thereby to determine a current status of mouthguard 400 and optionally one or more further mouthguard devices. This may be used to assess whether one or more mouthguards are in a fault state or the like. In some embodiments module 412 is configured to enable two-way communication with mouthguard 400, for example to enable remote switching of mouthguard 400 between multiple distinct operational settings (for example one optimised for impact detection, and one optimised for physical performance assessment).

A head impact detection and analysis module 413 is configured to process data derived from sensors of mouthguard 400 thereby to provide metrics representative of severity of an observed impact event. It will be appreciated that there are a range of technologies which may be used for this processing, for example using techniques to process linear and/or rotational acceleration, optionally using Al methods and/or benchmarking against existing data. In this example, module 413 operates in conjunction with a Finite Element Analysis (FEA) module 415. Module 413 is configured to process sensor data thereby to define a dosage input signal. This may include processing time correlated data from multiple sensors thereby to determine an acceleration value at a defined location (for example at the centre of gravity of the subject’s head, preferably based on transforms which are individually customised for the particular human subject based on their mouthguard and physical head configuration). This acceleration value is passed to FEA module 415, which performs analysis thereby to provide one or more metrics representative of predicted effect of the acceleration to the subject’s brain, thereby to provide data which assists in understanding anticipated severity of a head impact.

A VR system integration module 414 is configured to interact with a VR system that delivers tests as described further above. In some embodiments this includes providing instructions thereby to control delivery of tests. In some embodiments this includes receiving outputs representative of testing results. A VR/HID analysis module is configured to combine data derived from mouthguard 400 and from the VR system testing in relation to one or more impact events.

### Conclusions and Interpretation

It will be appreciated that the disclosure above provides improved technology for assessment of cognitive function. This includes technology which allows for improved concussion management in sports players via integrated impact detection and head injury assessment.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Various aspects of the present disclosure may be embodied as a program, software, or computer instructions embodied in a computer or machine usable or readable medium, which causes the computer or machine to perform the steps of the method when executed on the computer, processor, and/or machine. A program storage device readable by a machine, tangibly embodying a program of instructions executable by the machine to perform various functionalities and methods described in the present disclosure is also provided.

A system and method of the present disclosure may be implemented and run on a general-purpose computer or special-purpose computer system. The terms “computer system” and “computer network” as may be used in the present application may include a variety of combinations of fixed and/or portable computer hardware, software, peripherals, and storage devices. The computer system may include a plurality of individual components that are networked or otherwise linked to perform collaboratively, or may include one or more stand-alone components. The hardware and software components of the computer system of the present application may include and may be included within fixed and portable devices such as desktop, laptop, and/or server. A module may be a component of a device, software, program, or system that implements some “functionality”, which can be embodied as software, hardware, firmware, electronic circuitry, or etc.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

It should be appreciated that in the above description of exemplary embodiments of the invention, various features of the invention are sometimes grouped together in a single embodiment, FIG., or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. A computer implemented method configured to enable assessment of a brain injury or other physiological condition, the method including:
maintaining access to computer executable code representative of a plurality of neurological tests that are renderable via hardware including a virtual reality system, wherein the plurality of neurological tests includes neurological tests belonging to a plurality of distinct test classes;
configuring the virtual reality system to deliver, to a subject, using the virtual reality system, a neurological assessment including a sequence constructed from the plurality of neurological tests, wherein the sequence of the neurological tests is defined thereby to sequentially provide tests belonging to different ones of the plurality of distinct test classes, thereby to deliver an increasing cognitive load;
obtaining subject performance data representative of performance of the subject in the neurological assessment; and
processing the subject performance data thereby to derive one or more measures representative of subject neurological conditions.

2. A method according to claim 1 wherein the processing the subject performance data includes identifying variations in performance attributable to increasing of cognitive loading.

3. A method according to claim 2 wherein identifying variations in performance attributable to increasing of cognitive loading includes comparing subject performance with a plurality of tests belonging to a particular one of the distinct test classes which are delivered non-adjacently with respect to the sequence.

4. A method according to claim 2 wherein identifying variations in performance attributable to increasing of cognitive loading includes comparing subject performance with a first test belonging to a particular one of the distinct test classes with a second test belonging to the same particular one of the distinct test classes, wherein the second test is delivered subsequent to the first test non-adjacently with respect to the sequence.

5. A method according to any preceding claim wherein the plurality of distinct test classes includes one or more a test classes defined by defined forms of memory test.

6. A method according to claim 5 wherein the defined forms of memory test includes: immediate memory, working memory, and delayed memory tests.

7. A method according to any preceding claim wherein the plurality of distinct test classes include a class defined by a defined form of short-term memory test.

8. A method according to claim 7 wherein the defined form of short-term memory test includes a list item recollection exercise delivered via the virtual reality system.

9. A method according to any preceding claim wherein the plurality of distinct test classes include a class defined by a defined form of long-term memory test.

10. A method according to claim 9 wherein the defined form of memory test includes a list item recollection exercise delivered via the virtual reality system, wherein the list is presented preceding one or more tests of other test classes, and recollection tested following the one or more tests of other test classes.

11. A method according to any preceding claim wherein the plurality of distinct test classes include a class defined by a defined form of vestibular system test.

12. A method according to claim 11 wherein the defined form of vestibular system test includes a virtual reality game for which performance is related to gaze control and/or saccades, wherein the subject stands on a computerised balance board during the test.

13. A method according to any preceding claim wherein the plurality of distinct test classes include a class defined by a defined form of reaction time test.

14. A method according to claim 13 wherein the defined form of reaction time test includes an auditory reaction time test.

15. A method according to any preceding claim wherein the plurality of distinct test classes include a class defined by a defined form of ocular reaction time test.

16. A method according to claim 15 wherein the defined form of ocular reaction time test includes a test in which the VR system displays to the subject a moving object, and the subject is instructed to track that object with a stationary head, and provide a defined input upon the object performing a specified change in behaviour.

17. A method according to any preceding claim wherein the plurality of distinct test classes include a class defined by a defined form of vestibular system test in which the subject is instructed to shift balance on an computerised balance board.

18. A method according to any preceding claim wherein the plurality of distinct test classes include a class defined by a defined form of executive cognitive function test.

19. A method according to any preceding claim wherein the sequence constructed from a subset of the plurality of neurological tests is a sequence which includes a sub-sequence including three or more of the following thereby to increase cognitive loading through the sub-sequence: a memory test; a vestibular system test; a reaction time test; and an executive cognitive function test.

20. A method according to any preceding claim wherein the sequence is selected based on input data representative of an observed or suspected traumatic event involving the subject.

21. A method according to claim 20 wherein the input data representative of an observed or suspected traumatic event involving the subject includes input data based on measurements made by an instrumented mouthguard device.

22. A method according to claim 20 wherein the input data representative of an observed or suspected traumatic event involving the subject includes input data based on operation of a computerised brain model.

23. A method according to claim 21 wherein the computerised brain model is a Finite Element Analysis (FEA) model.

24. A method according to claim 23 wherein the FEA model is configured to operate based on input device from an instrumented mouthguard device.

25. A method according to any preceding claim wherein the one or more measures representative of subject neurological conditions are combined with data derived from instrumented observation of a traumatic event.

26. A method according to claim 25 wherein the instrumented observation of a traumatic event is observed via an instrumented mouthguard device.

27. A method according to claim 25 wherein the data derived from instrumented observation of a traumatic event includes output from a computerised brain model.

28. A method according to claim 27 wherein the computerised brain model is a Finite Element Analysis (FEA) model.

29. A method according to claim 28 wherein the FEA model is configured to operate based on input device from an instrumented mouthguard device.

30. A method according to any preceding claim wherein the one or more measures representative of subject neurological conditions are compared with benchmarked measures representative of subject neurological conditions for the subject.

31. A method for assessing a brain injury, the method including:
accessing a first data set representative of an observed traumatic event, wherein the first data set is generated in response to data derived from one or more subject-worn motion sensors;
accessing a second data set representative of neurological conditions following the observed traumatic event, wherein the second data set is generated in response to data derived from subject performance data in a neurological assessment delivered by a virtual reality system;
processing a combination of data from the first data set and the second data set thereby to define a third data set representative of an enhanced brain injury assessment.

32. A method according to claim 31 wherein the one or more subject-worn motion sensors are provided by an instrumented mouthguard device.

33. A method according to claim 31 wherein the first data set includes a metric derived from processing of data provided by the instrumented mouthguard device.

34. A method according to claim 31 wherein the first data set includes output from a brain model that is executed base on the data derived from one or more subject-worn motion sensors.

35. A method according to claim 34 wherein the brain model is a FEA model.

36. A method according to claim 35 wherein processing a combination of data from the first data set and the second data set includes identifying a correlation between an output of the FEA model and performance in the neurological assessment.

37. A method according to claim 36 wherein identifying a correlation between an output of the FEA model and performance in the neurological assessment includes benchmarking against prior results for different subjects.

38. A method according to claim 36 wherein identifying a correlation between an output of the FEA model and performance in the neurological assessment includes benchmarking against prior results for the same subject.

39. A method according to claim 31 wherein the third data set includes a metric representative of a deviation between: (i) expected performance in the neurological assessment based on the observed traumatic event; and (ii) actual performance in the neurological assessment based on the observed traumatic event.

40. A method according to claim 31 wherein the third data set is used to test and/or validate, via the second data set, a hypothesis as to the nature of a brain injury made based on the first data set.

41. A method for assessing a brain injury, the method including:
accessing a first data set representative of an observed traumatic event, wherein the first data set is generated in response to data derived from one or more subject-worn motion sensors;
based on the first data set, configuring a virtual reality system to deliver a neurological assessment having defined parameters to the subject, and in response define a second data set representative of subject performance in the assessment; and
performing a brain injury assessment based on a combination of the first data set and the second data set.

42. A method according to claim 41 wherein the one or more subject-worn motion sensors are provided by an instrumented mouthguard device.

43. A method according to claim 41 wherein the first data set includes a metric derived from processing of data provided by the instrumented mouthguard device.

44. A method according to claim 41 wherein the first data set includes output from a brain model that is executed base on the data derived from one or more subject-worn motion sensors.

45. A method according to claim 44 wherein the brain model is a FEA model.

46. A method according to claim 45 wherein the neurological assessment has one or more parameters selected based on an output of the FEA model.

47. A method according to claim 46 wherein the one or more parameters include a sequencing of sub-tests belonging to distinct classes.

48. A method according to claim 46 including identifying a correlation between an output of the FEA model and performance in the neurological assessment.

49. A method according to claim 41 including defining a measure representative of a deviation between: (i) expected performance in the neurological assessment based on the observed traumatic event; and (ii) actual performance in the neurological assessment based on the observed traumatic event.

50. A method according to claim 41 including performing a process thereby to test and/or validate, via the second data set, a hypothesis as to the nature of a brain injury made based on the first data set.
